(19) United States
(12) Patent Application Publication
(10) Pub. No.: US 2020/0337545 A1
(43) Pub. Date: Oct. 29, 2020

(54) MIRROR ASSEMBLY FOR USE IN DENTISTRY

(71) Applicant: DENTALEER LTD., Lod (IL)

(72) Inventor: SHLOMO BERKOVIC, HERTZLIYA (IL)

(21) Appl. No.: 16/919,837

(22) Filed: Jul. 2, 2020

Related U.S. Application Data
(63) Continuation-in-part of application No. PCT/IL2019/050001, filed on Jan. 1, 2019.
(60) Provisional application No. 62/612,731, filed on Jan. 2, 2018.

(51) Int. Cl. A61B 1/247; A61B 1/06

(52) U.S. Cl. CPC A61B 1/247; G10L 15/265; A61B 1/0684

(57) ABSTRACT

A dental mirror assembly comprises a handle that allows a practitioner to hold the system and manipulate it in the oral cavity of a patient. A shaft assembly connects a motor within the handle to a gear box that transfers angular momentum to the mirror. The power to spin the mirror flows from the motor within the handle to the shaft. From there, the power flows to a bevel gear and to then to a spinning mechanism, to spin the mirror. Switching mechanisms are used to switch the spinning mechanism.

### CROSS-REFERENCE TO RELATED APPLICATIONS

This application is a continuation-in-part of PCT Patent Application No. PCT/IL2019/050001, filed Jan. 1, 2019, and claims priority from U.S. Provisional Patent Application Ser. No. 62/612,731, filed Jan. 2, 2018, entitled “Mirror Assembly for Use in Dentistry,” each of which is incorporated herein by reference.

### TECHNICAL FIELD

The present invention relates to appliances useful in dentistry.

### BACKGROUND ART

The dentist's work entails carrying out surveying and active mechanical/surgical intervention in the patients' oral cavity. Small minors handled by the dentists and auxiliary staff members assist in viewing the nooks and crevices abundant in the oral cavity and more specifically in and around teeth/damaged teeth, jaws and gums.

The oral cavity, especially during dental treatment is a wet, humid warm environment which is conducive to obscurity of the mirror. US patent application 2016/0360957 discloses a dentists' mirror with a rotating mechanism which causes the mirror to rotate, in order to rid it of debris and humidity from the reflective surface. The rotation is brought about by power of the suction mechanism that dentists apply. In US patent application 2004/0076019 a rotatable dental mirror is disclosed in which the driving means supports the mirror directly from below.

The present application presents structurally and functionally leveraged minor for ameliorating the effective functionality of a dentist's mirror during treatment of patients.

### BRIEF SUMMARY OF THE INVENTION

According to a first aspect of the present disclosed subject matter, it is provided a self-cleaning dental mirror assembly comprising: a handle harboring a motor; a rotating mirror having a reflecting surface; a gear box connected to said rotating mirror, for rotating the rotating mirror; a shaft subassembly connecting the motor within the handle and the gear box; and a bevel gear within said gear box, wherein the bevel gear is facing the reflecting surface of the rotating mirror.

In accordance with another referred embodiment, the dental mirror further comprising a buttery within the handle.

In accordance with another referred embodiment, the dental mirror further comprising a switching mechanism for activating the motor to rotate the mirror.

In accordance with another referred embodiment, the dental mirror further comprising a switch on the handle for activating and deactivating the switching mechanism.

In accordance with another referred embodiment, the dental mirror further comprising a utterance recognition switching for activating and deactivating the switching mechanism using voice commands.

In accordance with another referred embodiment, the dental mirror further comprising a microphone located in the handle for receiving the voice commands.

In accordance with another referred embodiment, the dental mirror further comprising an automatic switch for detecting contamination on the reflective surface of the mirror, and automatically activating the switching mechanism when the reflective surface is unclean.

In accordance with another referred embodiment, wherein the automatic switch comprises a LED for emitting light; a photo-detector detecting the portion of the light emitted from the LED and reflected to the photo-detector from the mirror; and a processor for determining the portion of the light detected by the light detector and automatically activating the switching mechanism if the portion of the light detected by the light detector is below a predetermined threshold.

In accordance with another referred embodiment, wherein the LED and the photo-detector are located on the handle.

In accordance with another referred embodiment, wherein the LED emits infra-red light having wavelength that is absorbed by water.

In accordance with another referred embodiment, wherein the LED is aimed at the reflecting surface of the mirror, such that contamination of the mirror would cause the portion of the light detected by the light detector to be below a predetermined threshold, thus causes activation of the switching mechanism.

In accordance with another referred embodiment, wherein the gear box is a speed increasing gear.

In accordance with another referred embodiment, wherein the dental mirror assembly comprises a detachable section that is separated from the handle.

In accordance with another referred embodiment, wherein the detachable section comprises the rotating mirror and the gear box that are housed in a housing.

In accordance with another referred embodiment, wherein a shaft subassembly is a part of the detachable section.

In accordance with another referred embodiment, wherein the housing has dimensions similar to the dimensions of a standard dental mirror.

In accordance with another referred embodiment, wherein a thickness of the housing is less than 4 mm.

Unless otherwise defined, all technical and scientific terms used herein have the same meaning as commonly understood by one of ordinary skill in the art to which this disclosed subject matter belongs. Although methods and materials similar or equivalent to those described herein can be used in the practice or testing of the present disclosed subject matter, suitable methods and materials are described below. In case of conflict, the specification, including definitions, will control. In addition, the materials, methods, and examples are illustrative only and not intended to be limiting.

### BRIEF DESCRIPTION OF THE DRAWINGS

Some embodiments of the disclosed subject matter described, by way of example only, with reference to the accompanying drawings. With specific reference now to the drawings in detail, it is stressed that the particulars shown are by way of example and for purposes of illustrative discussion of the preferred embodiments of the present disclosed subject matter only, and are presented in the cause of providing what is believed to be the most useful and readily understood description of the principles and conceptual aspects of the disclosed subject matter. In this regard, no attempt is made to show structural details of the disclosed subject matter in more detail than is necessary for a fundamental understanding of the disclosed subject matter, the description taken with the drawings making apparent to those skilled in the art how the several forms of the disclosed subject matter may be embodied in practice.

In the drawings:

FIG. 1 is an isometric schematic general view of a mirror assembly of the invention, wherein the mirror is indirectly attached to the handle.

FIG. 2 is an isometric view of the mirror assembly, handle not shown.

FIG. 3 is a block diagram illustrating the flow of angular momentum in a device according to the invention.

FIG. 4A is an isometric schematic illustration of the gears facilitating the spinning of the mirror.

FIG. 4B is an isometric schematic illustration of the bevel gear and immediate mechanical parts, detailed window of FIG. 4A.

FIG. 4C is an isometric schematic illustration of the detachable mirror assembly, already seen in FIGS. 2, 4A and 4B.

FIG. 5 is a block scheme illustrating the functional connections between the components of the automatic mechanism for activating the spinning mechanism of the minor (AMAM).

FIG. 6 is a sequence scheme illustrating the main modules through which audio signal undergoes to ultimately activate the motor driver.

FIG. 7 is a block scheme describing the alternative switching mechanisms and their respective connections with the spinning mechanism/mirror.

### DISCLOSURE OF THE INVENTION

Before explaining at least one embodiment of the disclosed subject matter in detail, it is to be understood that the disclosed subject matter is not limited in its application to the details of construction and the arrangement of the components set forth in the following description or illustrated in the drawings. The disclosed subject matter is capable of other embodiments or of being practiced or carried out in various ways. Also, it is to be understood that the phraseology and terminology employed herein is for the purpose of description and should not be regarded as limiting. The drawings are generally not to scale. For clarity, non-essential elements were omitted from some of the drawings.

The terms “comprises”, “comprising”, “includes”, “including”, and “having” together with their conjugates mean “including but not limited to”. The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosed subject matter may be presented in a range format. It should be understood that the description in range format is merely for convenience and brevity and should not be construed as an inflexible limitation on the scope of the disclosed subject matter. Accordingly, the description of a range should be considered to have specifically disclosed all the possible sub-ranges as well as individual numerical values within that range.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination or as suitable in any other described embodiment of the disclosed subject matter. Certain features described in the context of various embodiments are not to be considered essential features of those embodiments, unless the embodiment is inoperative without those elements.

According to a preferred embodiment of the present subject matter, a self-cleaning dental mirror assembly is provided that comprises a handle harboring a motor; a rotating mirror having a reflecting surface; a gear box connected to said rotating mirror, for rotating the rotating mirror; a shaft subassembly connecting the motor within the handle and the gear box; and a bevel gear within said gear box, wherein the bevel gear is facing the reflecting surface of the rotating mirror.

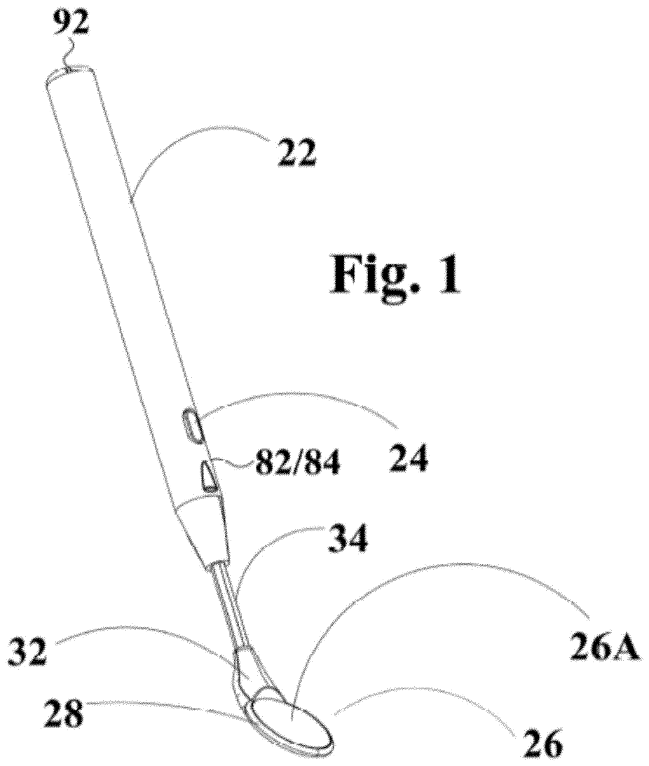

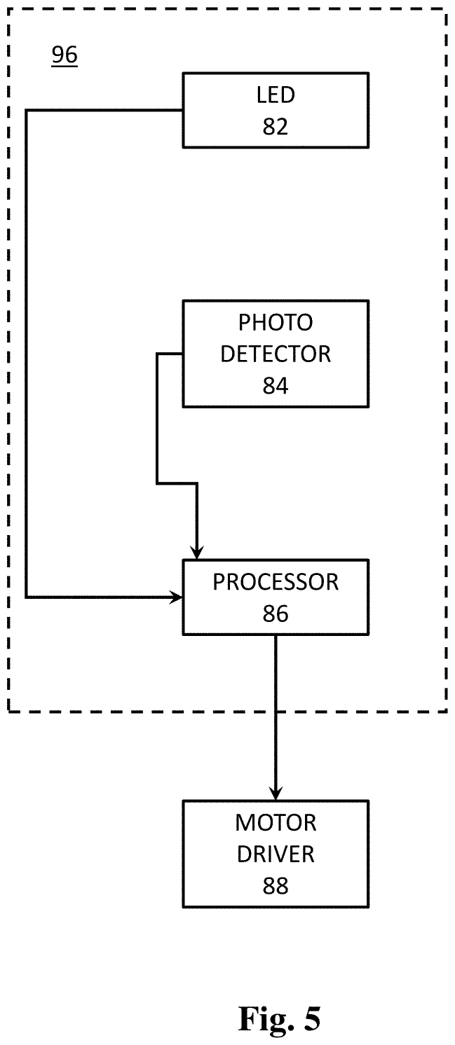

The mirror assembly 1 in accordance with the present invention is referred to in FIG. 1. Handle 22 is a module that allows the practitioner to hold the system and manipulating it within the oral cavity. It typically harbors a battery and a rotating electric motor (not seen in this figure). On/off Switch 24 facilitates activation or deactivation of the motor inside the handle. Mirror 26 has a reflecting side 26A shown in the figure. Mirror 26 is encased within mirror housing 28. Gearbox 32 is shown adjacent to mirror housing 28. A drive shaft subassembly 34 connects gearbox 32 to handle 22, or functionally speaking, to the motor concealed within handle 22.Also seen in FIG. 1 are the openings 92 for a microphone and the infra-red (IR) elements 82/84 of light reflection switching 96 to be detailed in FIGS. 5 and 7. The LED emits infra-red light having wavelength that is absorbed by water.

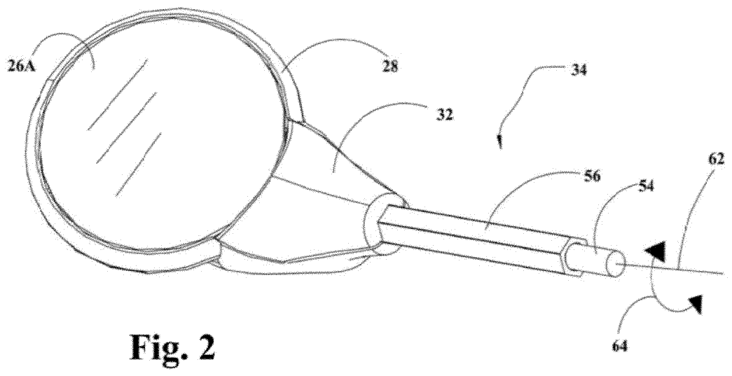

More structural features and functional properties are explained with reference to FIG. 2 which shows the detachable section accordance with the present invention. Mirror 26 has its reflective side 26A facing outwardly from housing 28. Gearbox 32 is located between mirror housing 28 and shaft subassembly 34. Here it can be seen that the shaft subassembly 34 includes shaft 54 sheathed by shaft cover 56. Shaft 54 is resolvable around axis 62 in either spin direction as indicated by double headed arrow 64.

### The Power Transfer System Including Gear

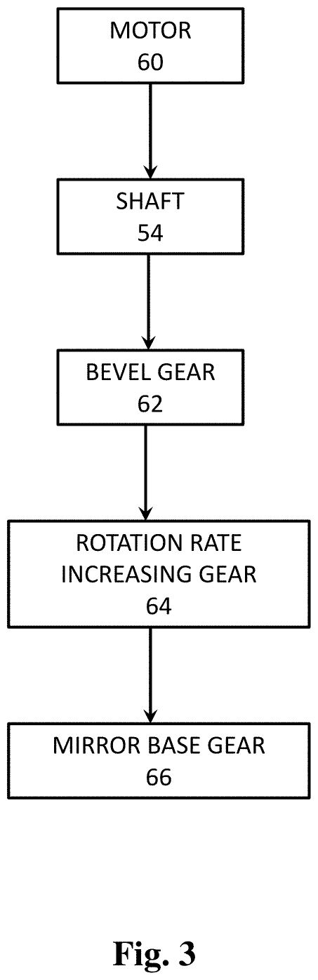

In accordance with the present invention, spinning of the mirror that takes effect in order to clear it of humidity and/or debris, is brought about by the transfer of angular momentum flowing from a motor towards the mirror. The motor, located in the handle, rotates a shaft 54, which passes on the angular momentum as can be seen in FIG. 3. Motor 60 transfers angular momentum to shaft 54, further transferring angular momentum to bevel gear 62. The angular momentum is further transferred to another gear that may optionally be a rotation rate increasing gear 64. Finally, the gear 66 at the base of the mirror receives the angular momentum for providing the desired power to spin the mirror. However, it should be pointed out that gears 64 and 66 are representative of several optional combinations of two to several more gears that can induce a desirable rotational velocity (spin) in the mirror.

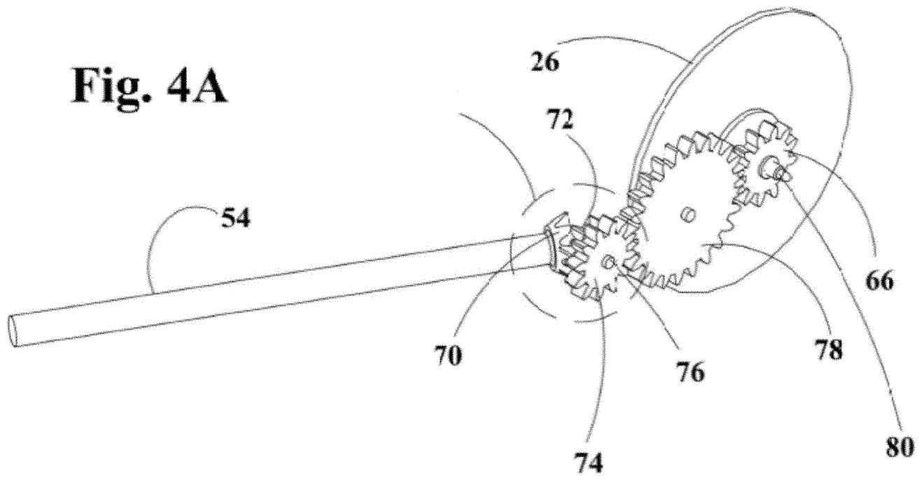

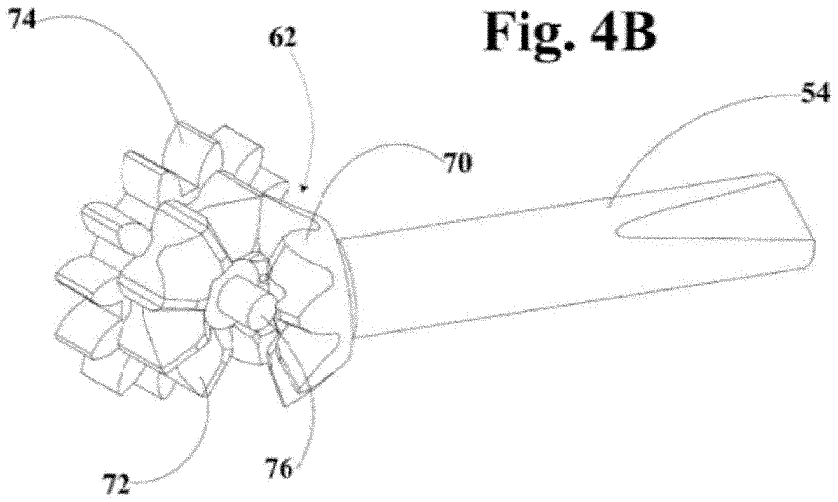

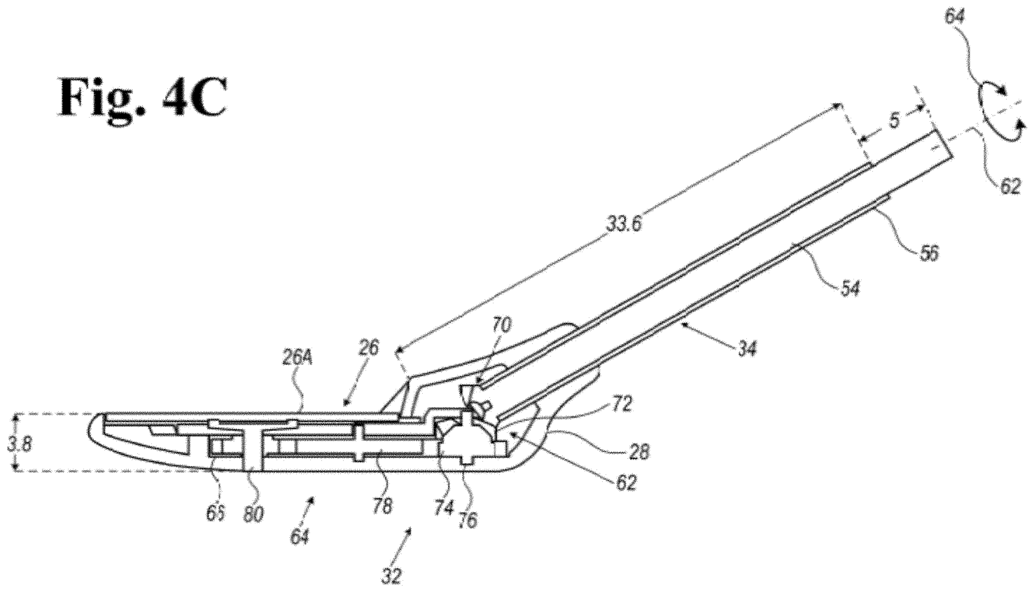

Structurally, a preferred angular momentum train is shown in FIGS. 4A and 4B. Shaft 54 has a conical gear 70 at its terminus that interacts with gear 72 (mostly hidden in this figure) having a common axis 76 with gear 74. Gear 74 interacts with gear 78. Gear 78 interacts also with gear 66 which is attached to the axis 80 of mirror 26. In FIG. 4B, the bevel gear and surrounding components of the angular momentum train are shown in more detail. Shaft 54 revolves, transferring angular momentum to bevel gear 62, consisting of two conical gear wheels. Conical gear wheel 70 is engaged with conical gear wheel 72, while axis 76 transfers the angular momentum to gear 74.

FIG. 4C is an isometric schematic illustration of the detachable section of the mirror assembly 1 already disclosed in of FIGS. 2, 4A and 4B. FIG. 4C, is merely for better showing some of its mechanical parts and measurements, and illustrates how the positioning of gear box 32 elements within the housing 28 allows creating a rotating mirror having typical small dimensions (marked in mm in the figure) similar to the dimensions of a standard dental mirror. Especially, the thickness of the housing 28 is kept to be less than 4 mm but preferably 3.8mm so as to maintain the regular dimensions the dentist is used to. The angle between the housing of the mirror and the shaft is also maintained as in the standard mirrors. For example, the conical gear 70 which interacts with gear 72 (already seen in FIGS. 4A and 4B) are facing the reflective face 26A of mirror 26 and thus can fit within the mirror housing 28.

### Mirror Maintenance

In such a case as the mirror is used, damaged or worn out, the detachable part 2 as shown in FIG. 2 can be pulled out of the handle 22 and be replaced or be sterilized and re-used. It should be noted at this point that sheath 56 may or may not be replaced, and is made to envelop shaft 54 without engaging with it in order to allow free spin within the sheath.

### Mirror Activation Automation

The dental mirror assembly is further comprising an automatic switch for detecting contamination on the reflective surface of the mirror, and automatically activating the switching mechanism when the reflective surface is unclean.

The automatic switch comprises a LED for emitting light; a photo-detector detecting the portion of the light emitted from the LED and reflected to the photo-detector from the mirror; and a processor for determining the portion of the light detected by the light detector and automatically activating the switching mechanism if the portion of the light detected by the light detector is below a predetermined threshold.

As the mirror gets damp and or soiled upon use, the user is expected to activate the spinning mechanism by turning on the on/off switch 24 in order to clear the mirror. A schematic block diagram describing the AMAM is depicted in FIG. 5 An alternative switching mechanism is an automatic one. Such an automatic mirror activation mechanism (AMAM) is an electro-optical module that includes a lighting element 82 (typically a LED, light emitting diode) located preferably on handle 22. The emitted light is propagated from the light source element to the reflective face 26A of the mirror. A photo-detector 84 located possibly in the vicinity of the LED 82, such as on handle 22 aims at the reflective mirror 26A. A certain portion of the light energy emanating from the LED 82 is received by photo-detector 84. Processor 86 calculates the ratio, and if the proportion of received power relative to the power of the beam is too low, and preferably below a predetermined value, the processor would switch the mirror driver 88 to start spinning the mirror automatically. In some embodiment, the light source of the AMAM such as lighting element 82 is an Infrared source, that is especially more sensitive to optical absorption by water relative to light in the visible wavelengths.

### Audio Switching

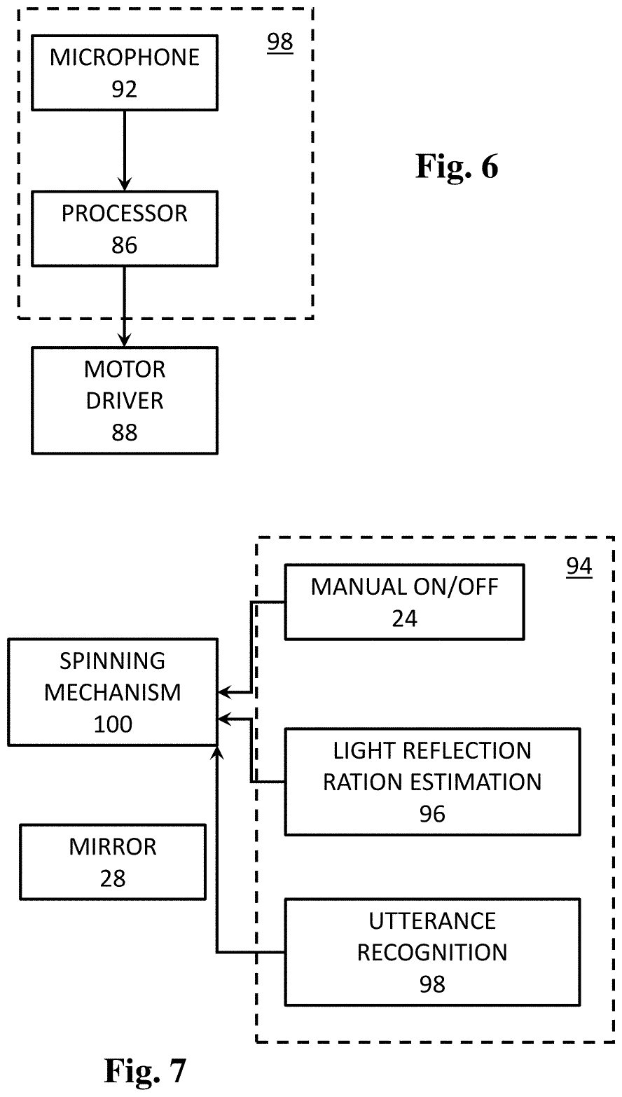

The LED is aimed at the reflecting surface of the mirror, such that contamination of the minor would cause the portion of the light detected by the light detector to be below a predetermined threshold, thus causes activation of the switching mechanism. A form of sound or word recognition module can be inserted in the dental mirror assembly to pick up and recognize words or utterances issued by the practitioner in order to switch on or off the spinning mechanism. As the case is with known voice recognition mechanisms, the mechanism has to go through a training session. As can be seen in FIG. 6, microphone 92 functionally connects to processor 86, after the signal has been digitized and prepared for acceptance by the processor. Once the signal has been analyzed and interpretation achieved, a positive outcome will activate the motor driver 88. The practitioner may decide to evoke the system by an utterance or a specific sound so that instances of false alarms are minimized. Such a module is typically harbored in the handle.

In FIG. 7, the optional switching mechanisms 94 are shown as they refer to mirror spinning. Switching mechanisms are either manual switching 24, light reflection switching 96, utterance recognition switching 98. These are all mutually exclusively activated, effecting spinning mechanism 100 to subsequently spin mirror 26.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A self-cleaning dental mirror assembly comprising:
a handle harboring a motor;
a rotating mirror having a reflecting surface;
a gear box connected to said rotating mirror, for rotating the rotating mirror;
a shaft subassembly connecting the motor within the handle and the gear box; and
a bevel gear within said gear box, wherein the bevel gear is facing the reflecting surface of the rotating minor.

2. The dental mirror assembly as claimed in claim 1, further comprising a buttery within the handle.

3. The dental mirror assembly as claimed in claim 1, further comprising a switching mechanism for activating the motor to rotate the mirror.

4. The dental mirror assembly as claimed in claim 3, further comprising a switch on the handle for activating and deactivating the switching mechanism.

5. The dental mirror assembly as claimed in claim 3, further comprising an utterance recognition switching for activating and deactivating the switching mechanism using voice commands.

6. The dental mirror assembly as claimed in claim 5, further comprising a microphone located in the handle for receiving the voice commands.

7. The dental mirror assembly as claimed in claim 3, further comprising an automatic switch for detecting contamination on the reflective surface of the mirror, and automatically activating the switching mechanism when the reflective surface is unclean.

8. The dental mirror assembly as claimed in claim 7, wherein the automatic switch comprises:
an LED for emitting light;
a photo-detector detecting the portion of the light emitted from the LED and reflected to the photo-detector from the mirror; and
a processor for determining the portion of the light detected by the light detector and automatically activating the switching mechanism if the portion of the light detected by the light detector is below a predetermined threshold.

9. The dental mirror assembly as claimed in claim 8, wherein the LED and the photo-detector are located on the handle.

10. The dental mirror assembly as claimed in claim 9, wherein the LED emits infra-red light having wavelength that is absorbed by water.

11. The dental mirror assembly as claimed in claim 9, wherein the LED is aimed at the reflecting surface of the mirror, such that contamination of the mirror would cause the portion of the light detected by the light detector to be below a predetermined threshold, thus causes activation of the switching mechanism.

12. The dental mirror assembly as claimed in claim 1, wherein the gear box is a speed increasing gear.

13. The dental mirror assembly as claimed in claim 1, wherein the dental mirror assembly comprises a detachable section that is separated from the handle.

14. The dental mirror assembly as claimed in claim 13, wherein the detachable section comprises the rotating mirror and the gear box that are housed in a housing.

15. The dental mirror assembly as claimed in claim 13, wherein a shaft subassembly is a part of the detachable section.

16. The dental mirror assembly as claimed in claim 14, wherein the housing has dimensions similar to the dimensions of a standard dental mirror.

16. The dental mirror assembly as claimed in claim 14, wherein a thickness of the housing is less than 4 mm.
